(19) United States
(12) Patent Application Publication
(10) Pub. No.: US 2014/0227329 A1
(43) Pub. Date: Aug. 14, 2014

(54) MICROCAPSULES HAVING AN ENVELOPE COMPOSED ESSENTIALLY OF SILSESQUIOXANE HOMOPOLYMERS OR COPOLYMERS

(71) Applicant: MICROCAPSULES TECHNOLOGIES, Puiseaux (FR)

(72) Inventor: Gerard Daniel HABAR, Bourron-Marlotte (FR)

(73) Assignee: MICROCAPSULES TECHNOLOGIES, Puiseaux (FR)

(21) Appl. No.: 14/255,045

(22) Filed: Apr. 17, 2014

Related U.S. Application Data
(62) Division of application No. 13/124,969, filed on Apr. 19, 2011, now abandoned.

(30) Foreign Application Priority Data
Oct. 20, 2008 (FR) 0857111

(51) Int. Cl. A61K 8/11; A61Q 19/08; A61Q 19/00; A61K 8/365

(52) U.S. Cl. CPC A61K 8/11; A61K 8/365; A61Q 19/08; A61Q 19/00; A61K 2008/115; A61K 2800/95
USPC 424/401; 512/4

(57) ABSTRACT

A microcapsule having a reservoir that includes a core containing at least one active principle, the core being surrounded by a polymer shell, is presented. The polymer shell contains 50% to 100% by weight of a silsesquioxane compound with respect to the total weight of the shell. A process for the manufacture of the microcapsules is also presented. The microcapsules containing active principles can be used in cosmetic products.

### BACKGROUND OF THE INVENTION

The present invention relates to microcapsules of core/shell type or reservoir microcapsules each comprising a core (generally liquid) surrounded by a shell (generally solid) composed essentially of silsesquioxane homopolymers or copolymers.

The present invention also relates to the process for the manufacture of the abovementioned microcapsules and to their use in manufacturing cosmetic products.

### DESCRIPTION OF THE PRIOR ART

Microcapsules including a lipophilic or hydrophilic active principle are used in numerous fields, for example in the fields of cosmetics or pharmaceuticals. Active principles, such as fragrance, UV screening agents or medicaments, can be inserted in microcapsules, in order to be protected therein, and then slowly released.

There exist two types of microcapsules, depending on the hydrophilic or lipophilic nature of the active principle present in the microcapsules. Thus, when the microcapsules comprise an aqueous internal phase, the continuous phase is organic and, when they comprise an organic internal phase, the continuous phase is aqueous.

Numerous microcapsules have been developed in the prior art, in particular microcapsules based on silsesquioxane, which is an inexpensive and readily available compound exhibiting numerous advantages. It exhibits a good thermal and mechanical stability, it is resistant to light and it is biologically inactive. It is consequently well tolerated by the skin, in particular human skin.

In a known way, silsesquioxanes refer to the general empirical formula R—SiO_{3/2}, where Si is the element silicon, O is oxygen and R is an alkyl, alkenyl, aryl or arylene group. Silsesquioxanes are generally obtained by hydrolysis and condensation of organotrialkoxysilanes corresponding to the general empirical formula: R—Si(OR₁)₃, where R is as defined above and R₁ is a generally alkyl radical.

The document U.S. Pat. No. 3,257,330 describes in particular a process for the manufacture of a particle based on a colored gel comprising an organopolysiloxane as matrix. However, when an alkoxysilane exhibiting a hydrophobic organic group, such as methyltriethoxysilane, is used as starting material for the matrix (hydrolysis reaction), the polymer composition then forms a deposit in an aqueous solution.

Consequently, it is difficult to manufacture a microcapsule while incorporating a hydrophobic core during the polymerization of a hydrolyzate with an alkoxysilane in an aqueous solution.

The document U.S. Pat. No. 3,551,346 describes, in its prior art, a process for the manufacture of microcapsules in which a polysiloxane is synthesized from a trialkoxysilane. However, the shell of the microcapsules, which is composed of the polysiloxane, does not exhibit a sufficient resistance and a sufficient hardness to be suitable for the encapsulation of active principles. For this reason, the solution found by this document is that of manufacturing microcapsules comprising a wall having two layers.

As is indicated in this document, it is difficult at the present time to manufacture a microcapsule having just one shell based on organopolysiloxane.

Patent U.S. Pat. No. 6,251,313 describes microcapsules having an organopolysiloxane wall manufactured by polymerization in a basic medium in the presence of aminated silane monomers.

The disadvantage of such a process carried out in a basic medium is not only the presence of a residual porosity in the organopolysiloxane wall but also a yellowing of the microcapsules to light brought about by the amine groups present. Furthermore, the disadvantage of this technique in a basic medium is that the polymer being formed has straightaway a three-dimensional structure which rapidly stiffens and inevitably results in porous microcapsules.

Thus, the solutions found by the state of the art are to involve: either several monomers which have been specifically measured out, rendering the reaction complicated and expensive; or copolymers, which are difficult to synthesize, carrying long chains in order to render the structure flexible—however, in this case, the copolymers react slowly; or reducing the functionality of the monomers—however, in the latter case, the reactivity is reduced and the final structure is weakened.

The document EP 0 661 334 describes fine particles of silicone gum with a mean diameter of 0.1 μm to 100 μm comprising a coating based on polyorganosilsesquioxane resin, this coating representing from 1 to 500 parts by weight to 100 parts by weight of particles of silicone gum. This document describes a technique for grafting to solid particles. Specifically, the solid particles of silicone gum (cured silicone rubber) are covered with a polyorganosilsesquioxane resin by reacting (hydrolysis and condensation reaction) a trialkoxy-silane compound with an aqueous dispersion of silicone gum. In addition, with the process as described in this document, it is not possible to obtain microcapsules predominantly based on silsesquioxane since, according to this process, the alkoxysilanes polymerize not around the droplets of liquid active principles but polymerize in the form of small particles in the aqueous phase.

The document EP 1 426 100 describes particles formed of a polymer of silsesquioxane type, such as the phenyl-propylsilsesquioxane of example 1, within which an active principle (hair dye, UV-A or UV-B screening agents, flavonoids, and the like) is absorbed. This document thus does not describe reservoir microcapsules exhibiting a core (lipophilic phase or aqueous phase) surrounded by an external shell (polymer).

The publication “Core/Shell Silica-Based in situ Microencapsulation: A Self-Templating Method” by Bok Yeop Ahn describes microcapsules comprising a lipophilic active core (liquid) and a solid coating composed of silica (SiO₂) and of (RSiO_{1.5})₁₋ₓ-(SiO₂)ₓ, R being an alkyl group and x ranging from 0.1 to 0.5. The silica is formed from tetraethoxysilane (TEOS) and the second compound (_{RSiO}_{1.5})₁₋ₓ-(SiO₂)ₓ is itself formed by a combination of Si(OR)₄ and of RSi(OR′)₃, such as methyltrimethoxysilane (MTMS). Consequently, in this document, the silsesquioxane compound is only an additive for supplementing the silica prepolymer. Furthermore, in the example, it does not represent more than 30% of the coating.

Likewise, the publication “Microencapsulation of Oil in Organically Modified Silicate Glass by Sol-Gel Process” by Sang I. Seok describes a process for the preparation of a microcapsule comprising a lipophilic core (xylene) and a shell based mainly on silica and on a compound of silsesquioxane type as additive. The first stage of the process of this document consists in:
hydrolyzing and condensing tetraethyl orthosilicate and methyltrimethoxysilane (MTMS) with deionized water, so as to form an oligomeric compound,
simultaneously removing the alcohol formed during the hydrolysis,
mixing, after cooling, the oligomeric compound obtained with a lipophilic compound: xylene (oily phase) with a doping agent, and
homogenizing the oily phase/oligomer mixture, so as to form a water-in-oil microemulsion.

The document EP 0 216 388 relates to a process for removing atmospheric pollutants (NOₓ, SO₂) starting from a gas.

None of these documents describes a reservoir microcapsule, the coating of which would be essentially based on a compound of silsesquioxane type.

### SUMMARY OF THE INVENTION:

The aim of the present invention is to provide a novel process for the manufacture of microcapsules and novel microcapsules including a lipophilic or hydrophilic active principle which avoid all or some of the abovementioned disadvantages.

A subject matter of the present invention is a reservoir microcapsule comprising a core comprising at least one active principle, said core being surrounded by a polymer shell, wherein said polymer shell is formed from 50 to 100% by weight of a compound of silsesquioxane type, with respect to the total weight of said shell.

A reservoir microcapsule (or core/shell microcapsule) comprises a core surrounded by a shell made of polymer. Generally, the core is more or less liquid and the coating is more or less solid. Thus, the microcapsule, as its name indicates, is composed of a capsule formed by a continuous shell made of silsesquioxane polymer (according to the present invention) surrounding a core itself composed of active principles. The aim of a reservoir microcapsule is to comprise, inside the polymer shell, which has to be solid and resistant, a core formed of active principles. This type of technology is different from the grafting technique, where the coating is grafted to solid particles (silicone gum examples), or the technique of matrix type, where active principles are absorbed on a solid polymer and there is no exterior shell (the polymer is synthesized and then the active principle is incorporated therein).

As the reservoir microcapsules according to the present invention comprise a wall essentially based on a compound of silsesquioxane type, they exhibit a sufficient strength and a sufficient leaktightness to be suitable for the encapsulation of lipophilic or hydrophilic active principles.

Preferably, the polymer compound of silsesquioxane type represents 70% or more by weight, with respect to the total weight of said shell.

Advantageously, the polymer compound of silsesquioxane type is R-SiO_{3/2}, where R is:
a substituted or unsubstituted alkyl radical having from 1 to 20 carbon atoms, such as methyl, ethyl, n-propyl, isopropyl, 1-n-butyl, 2-n-butyl, isobutyl, tert-butyl, n-pentyl, isopentyl, neopentyl, tert-pentyl, hexyl, such as n-hexyl, heptyl, such as n-heptyl, octyl, such as n-octyl or isooctyl, 2,2,4-trimethylpentyl, nonyl, decyl, dodecyl, octadecyl, cycloalkyl, such as cyclopentyl, cyclohexyl, cycloheptyl and methylcyclohexyl, aryl, such as phenyl, naphthyl, anthryl and phenanthryl, alkaryl, such as o-, m- and p-tolyl, xylyl and ethylphenyl, and aralkyl, such as benzyl, α-phenylethyl and β-phenylethyl, radicals,
an oxygen-comprising alkyl radical, such as methoxyethyl and ethoxyethyl,
a halogenated radical, such as chloropropyl, 3,3,3-trifluoro-n-propyl, 2,2,2,2′,2′,2′-hexafluoro-isopropyl, heptafluoroisopropyl or o-, m- and p-chloro-phenyl,
or an unsaturated radical, such as vinyl, 5-hexenyl, 2,4-divinylcyclohexylethyl, 2-propenyl, allyl, 3-butenyl, 4-pentenyl, ethynyl, propargyl and 2-propynyl.

Preferably, the active principle or principles are chosen from: fatty acids and alcohols, organic solvents, hydrocarbons, esters, silicone fluids and gums, vegetable oils and lipophilic or hydrophilic plant extracts, reactive or unreactive dyes as well as pigment dispersions, UV screening agents, vitamins and medicinally active molecules which are pure or in aqueous or organic solution, fragrances and flavorings, insecticides and repellants, catalysts, phase change materials, phenolic compounds, water, disinfecting agents, such as aqueous hydrogen peroxide solution, glutaraldehyde in solution, salts, amino acids, proteins, polypeptides, enzymes, DHA, saccharides and polysaccharides, amine salts or their mixtures.

Another subject matter of the present invention is a process for the manufacture of reservoir microcapsules as described above, comprising the stages consisting in:

(i) dispersing at least one lipophilic or hydrophilic active principle in a respectively aqueous or organic continuous phase, so as to respectively form an oil-in-water or water-in-oil emulsion or dispersion,

(ii) hydrolyzing a precursor of the polymer compound of silsesquioxane type and polymerizing it in situ in or on contact with the aqueous phase of the oil-in-water or water-in-oil dispersion or emulsion, so as to form a silsesquioxane homopolymer or copolymer,

wherein (iii) a compound chosen from:
a silicate which is preferably insoluble in water in the hydrolyzed state, such as poly(ethyl silicate),
a precursor of the polymer compound of silsesquioxane type,
or their mixtures,

is introduced into the organic phase of the microcapsules at the beginning of the hydrolysis and/or polymerization reaction,

so as to confer, on the polymerization or on the encapsulation, an interfacial nature favorable to the leaktightness of the microcapsules.

This is because the addition of one of these compounds (preferably insoluble silicate or precursor of the silsesquioxane polymer compound) to the organic phase of the mixture makes it possible to obtain microcapsules based essentially on silsesquioxane, whether in a basic medium or in an acidic medium.

Preferably, the polymerization stage is carried out in an acidic medium.

This is because the studies of the applicant company have shown, surprisingly and unexpectedly, that, if, during the manufacture of microcapsules, the silsesquioxane polymer or copolymer is synthesized in situ by hydrolysis and polymerization in an acidic medium, then it was possible to more easily obtain (compared to a basic medium) microcapsules having just one resistant and leaktight shell composed of polymer of silsesquioxane type.

Consequently, in view of the existing disadvantages in the manufacture of silsesquioxane-based microcapsules, a person skilled in the art would not have been inclined to carry out the hydrolysis and polymerization stages in an acidic medium. This is because, in the techniques described above, the hardening is always carried out by increasing the pH, bringing it into the basic region, where the polymerization crosslinkings are fast and complete.

Preferably, the pH during the polymerization is less than 6.

According to a first alternative embodiment, the pH lies between 3 and 5 during the hydrolysis and during the beginning of the polymerization and is then from 1 to 4, preferably from 1.5 to 2.5, up to the end of the polymerization.

According to a second alterative embodiment, the pH lies between 1 and 4 from the hydrolysis stage.

Advantageously, fluoride ions or one or more compounds comprising fluoride ions in their structure are present in the medium during the polymerization.

In particular, the fluoride ions are used in the presence of a compound carrying an amine functional group.

Advantageously, the pH at the end of the polymerization reaction has risen to between 5.5 and 8.5, preferably between 6 and 7.

Preferably, in the case of an oil-in-water emulsion, one or more silanes carrying hydrophilic groups are introduced after at least partial solidification of the wall of the microcapsules.

Advantageously, in the case of a water-in-oil emulsion, one or more silanes carrying lipophilic groups are introduced after at least partial solidification of the wall of the microcapsules.

According to the two characteristics above, optionally at least one silane carries cationic charges.

Advantageously, the temperature lies between 10° C. and 50° C. during the dispersion or hydrolysis stage and is then from 40° C. to 90° C. during the polymerization stage.

Preferably, the precursor of the polymer compound of silsesquioxane type is of the R—Si(R₁R₂R₃) type, where R is as defined above,

where R₁, R₂ and R₃ each independently denote an acetoxy, amino, acid, amide, oximino, chlorine or OR₄ group where

R₄is:
a substituted or unsubstituted alkyl radical having from 1 to 3 carbon atoms, such as, for example, methyl, ethyl, n-propyl or isopropyl radicals,
an oxygen-comprising alkyl radical, such as methoxyethyl and ethoxyethyl,
or an unsaturated radical, such as vinyl, 2-propenyl or allyl.

In particular, the precursor of the polymer compound of silsesquioxane type is methyltrimethoxysilane (MTMS), methyltriethoxysilane (MTES), methyltrichlorosilane or their mixtures.

Another subject matter of the present invention is the use of a reservoir microcapsule as described above in the manufacture of a cosmetic or pharmaceutical product exhibiting a UV screening agent.

### DETAILED DESCRIPTION OF THE INVENTION:

The oil-in-water and then water-in-oil encapsulation preparations will be presented below, followed by nonlimiting examples.

a) Oil-In-Water Encapsulation:

In the case of an oil-in-water encapsulation, a lipophilic internal phase (lipophilic active principles) is dispersed in an aqueous continuous phase.

Preparation of the Lipophilic Internal Phase:

In order to prepare a lipophilic internal phase, one or more lipophilic active principles are mixed.

The active principles, which also comprise fatty substances, are chosen, for example, from: antioxidants, agents for combating free radicals, melanin regulators, tanning accelerators, depigmenting agents, skin coloring agents, liporegulators, slimming agents, antiacne agents, antiseborrheic agents, antiaging agents, antiwrinkle agents, agents for combating UV radiation, keratolytic agents, anti-inflammatory agents, refreshing agents, healing agents, vasoprotective agents, antibacterial agents, antifungal agents, antiperspirants, deodorants, hair conditioners, immunomodulators, nourishing agents, essentials oils and fragrances.

Mention may more particularly be made, as examples of lipophilic active principles for the treatment of the skin and/or hair which can be used in the context of the present invention, of the following compounds: D-α-tocopherol, DL-α-tocopherol, D-α-tocopherol acetate, DL-α-tocopherol acetate, ascorbyl palmitate, vitamin F glycerides, vitamins D, in particular vitamin D₂ and vitamin D₃, retinol, retinyl esters (retinyl palmitate, retinyl propionate), 13-carotene, D-panthenol, farnesol, farnesyl acetate, oils rich in essential fatty acids, in particular jojoba oil and blackcurrant oil, 5-(n-octanoyl)salicylic acid, salicylic acid, alkyl esters of α-hydroxy acids, such as citric acid, lactic acid and glycolic acid, asiatic acid, madecassic acid, asiaticoside, total extract of Centella asiatica, β-glycyrrhetinic acid, α-bisabolol, ceramides, in particular 2-oleoylamino-1,3-octadecane, phytanetriol, milk sphingomyelin, phospholipids of marine origin rich in polyunsaturated essential fatty acids, ethoxyquin, rosemary extract, balm extract, quercetin, extract of dried microalgae (Algoxan Red, sold by Algatec), bergamot essential oil, octyl methoxycinnamate (Parsol MCX, sold by Givaudan-Roure), butylmethoxydibenzoylmethane (Parsol 1789, sold by Guivaudan-Roure), octyl triazone (Uvinul T150, sold by BASF), yellow, brown, black or red iron oxides, titanium oxides, which can be provided in the micrometric or nanometric form or in the coated form (for example coated by a perfluoroalkyl), 3-[3,5-di(tert-butyl)-4-hydroxybenzylidene]camphor, 2-(benzo-triazol-2-yl)-4-methyl-6-[3-[1,3,3,3-tetramethyl-1-[(trimethylsilyl)oxy]disiloxanyl]-2-methylpropyl]-phenol, perfluorinated oil (perfluorodecalin, perfluorooctyl bromide) or hyperoxygenated maize oil (Epaline 100, sold by Carilene).

In an alternative embodiment, it is possible to add, to this mixture of lipophilic active principles, a precursor of the polymer compound of silsesquioxane type.

Preferably, the precursor of the polymer compound of silsesquioxane type is of the R—Si(R₁R₂R₃) type in which

R represents a nonhydrolyzable radical and R₁, R₂ and R₃ represent hydrolyzable radicals.

R is in particular:
a substituted or unsubstituted alkyl radical having from 1 to 20 carbon atoms, such as, for example, methyl, ethyl, n-propyl, isopropyl, 1-n-butyl, 2-n-butyl, isobutyl, tert-butyl, n-pentyl, isopentyl, neopentyl, tert-pentyl, hexyl, such as n-hexyl, heptyl, such as n-heptyl, octyl, such as n-octyl or isooctyl, 2,2,4-trimethylpentyl, nonyl, decyl, dodecyl, octadecyl, cycloalkyl, such as cyclopentyl, cyclohexyl, cycloheptyl and methylcyclohexyl, aryl, such as phenyl, naphthyl, anthryl and phenanthryl, alkaryl, such as o-, m- and p-tolyl, xylyl and ethylphenyl, and aralkyl, such as benzyl, α-phenylethyl and β-phenylethyl, radicals,
an oxygen-comprising alkyl radical, such as methoxyethyl and ethoxyethyl,
a halogenated radical, such as chloropropyl, 3,3,3-trifluoro-n-propyl, 2,2,2,2′,2′,2′-hexafluoro-isopropyl, heptafluoroisopropyl or o-, m- and p-chloro-phenyl,
or an unsaturated radical, such as vinyl, 5-hexenyl, 2,4-divinylcyclohexylethyl, 2-propenyl, allyl, 3-butenyl, 4-pentenyl, ethynyl, propargyl and 2-propynyl; and R₁, R₂ and R₃ denote hydrolyzable groups, such as methoxy, ethoxy, propoxy, isopropoxy, methoxyethoxy, acetoxy, amino, acid, amide or oximino, or chlorine atoms.

The hydrolysis reactions result in the monomer R—Si(OH)₃.

Short chains, which give higher reaction rates, will be preferred.

The precursors exhibiting short chains will be used in preference as they give higher reaction rates.

Preferably, the precursor of the polymer compound of silsesquioxane type is methyltrimethoxysilane (MTMS), methyltriethoxysilane (MTES), methyltrichlorosilane or their mixtures. The advantage of these compounds is that they rapidly result, under appropriate conditions, in microcapsules having a hard wall which is highly resistant chemically and microbiologically and which is only very slightly porous.

It is also possible to add, to this mixture of lipophilic active principles, an organosilicate which preferably remains insoluble in the water in the hydrolyzed state, such as poly(ethyl silicate). In an alternative embodiment, this compound can also be introduced at the beginning of the hydrolysis and/or polymerization reaction. This technique makes it possible to better “anchor” the silsesquioxane being formed to the microcapsule and to also reduce the hydrophilicity of the combination.

These compounds, the poly(ethyl silicate) or the precursor of the polymer compound of silsesquioxane type (MTMS, MTES), when they are added to the lipophilic phase, make it possible, surprisingly, to give a partially interfacial nature to the polymerization.

According to the prior art, in an in situ encapsulation, the polymerization takes place in the aqueous phase. During the polymerization of monomers of organosilane type, this being done in order to form a polymer of silsesquioxane type (or other silicone), there is formation of R—Si(OH)₃, followed by polymerization with formation of a polymer which comprises many OH groups. As the reaction continues, the number of OH groups decreases. This polymer being formed is thus very hydrophilic at the start, and it thus has no tendency immediately to be deposited around the oil drops but has a tendency to remain in aqueous solution, giving very high viscosities which render the operations difficult, indeed even impossible. Furthermore, this polymer being formed is only deposited around the drops when it has become depleted in OH. The molecular weight of the polymer, its degree of polymerization and its degree of crosslinking are then such that it is not homogeneously deposited with the formation of a compact liquid layer. Consequently, a porous wall is formed.

The advantage of employing an organosilane monomer, such as MTES, or a water-insoluble prepolymer, such as poly(ethyl silicate) or other, in the oily phase is that reaction occurs at the interface between these organosilane monomers or prepolymers and the polymer which is formed in the water. Thus, the silsesquioxane polymer being formed is bonded to the oil drops and is deposited around them much more easily and much sooner.

Due to this, the encapsulation according to the process of the invention exhibits an interfacial nature which renders the microcapsules leaktight and resistant.

As mentioned above, a silicate which is preferably insoluble in water in the hydrolyzed state, such as poly(ethyl silicate), or a precursor of the polymer compound of silsesquioxane type, such as MTMS or MTES, which are capable of remaining in the oil, are more particularly suitable as, being in the oily phase, they will be hydrolyzed much less rapidly than the precursors present in the water. Furthermore, a certain amount of these compounds will be in a form already partially polymerized but not completely hydrolyzed (as the nonhydrolyzed groups will have a tendency to remain in the oil) and consequently will be able to react with the silsesquioxane precursor present in the aqueous phase and help it to be deposited around the oil drops.

This advantageous effect is also valid for a water-in-oil encapsulation.

Finally, it is also possible to add, to this mixture of lipophilic active principles, a lipophilic amine, such as a tributylamine or a dimethylbenzylamine. This amine will form, at the water/oil interface, a complex with the fluoride ions of the aqueous phase, which complex will catalyze the reaction by accentuating its interfacial nature.

This first mixture will become the lipophilic internal phase of the microcapsules.

Preparation of the Aqueous Continuous Phase:

The aqueous continuous phase comprises water and one or more acids, preferably weak acids, so that the pH is less than 6 and preferably lies between 3 and 5. These weak acids are, for example, acetic acid, formic acid or citric acid.

One or more precursors of polymer compounds of silsesquioxane type of the R—Si(R₁R₂R₃) type as described above are introduced into this acidic aqueous phase.

In order to promote the formation of the emulsion or to help keep it intact during encapsulation, it is possible to introduce a protective colloid into the continuous phase. This protective colloid can be chosen from the following list: cellulose derivatives, such as hydroxyethylcellulose, carboxyethylcellulose and methylcellulose, polyvinylpyrrolidones and vinyl-pyrrolidone copolymers, poly(vinyl alcohol)s which are hydrolyzed to a greater or lesser extent, and their copolymers, polymers of natural origin, such as gelatin, xanthan gum or gum arabic, alginates, pectins, starches and derivatives, casein and ionized polymers, such as polymers and copolymers of acrylic or methacrylic acid or polymers carrying sulfo groups. In addition, these colloids make it possible to obtain a particle size dispersion of the emulsion or of the dispersion which is not excessively broad and to reduce agglomerations during the polymerization of the shell.

Manufacture of the emulsion/dispersion, hydrolysis and beginning of polymerization:

The lipophilic internal phase is mixed with the aqueous continuous phase with stirring. According to another alternative embodiment, it is possible to wait for the hydrolysis of the precursors of the polymer compound of silsesquioxane type to take place before introducing the internal phase.

This addition takes place at a temperature lying between 10° C. and 50° C., preferably between 20° C. and 40° C.

This operation can be carried out using stirrers, homogenizers or rotor/stator turbine mixers. The rotational speed serves to regulate the size of the microcapsules, which will be adjusted generally to between 0.1 and 100 μm.

Surfactants can be used in order to facilitate this operation but are generally unnecessary. By way of example, it is possible to use: sorbitan or glycerol fatty acid esters which are oxyethylenated to a greater or lesser extent; polyoxyethylenated derivatives of phenols carrying fatty chains, amino or amido betaines carrying fatty chains, oxyethylenated fatty acid or fatty alcohol condensates, alkylarylsulfonates, fatty acid soaps, fatty sulfates and sulfonates, dialkyl sulfosuccinates, oxides of fatty amines, fatty imidazolines, fatty amido sulfobetaines, cationic emulsifiers, mono- or diethanolamides of fatty acids, dispersants of silicone type, such as dimethicone copolyols, or their mixtures.

The internal phase is present in the emulsion or the dispersion of the microcapsules at a level of 35 to 40% approximately.

At this stage, the walls of the microcapsules are liquid. The silsesquioxane precursor begins to surround the dispersed phase as it is hydrolyzed.

Continuation and acceleration of the polymerization:

After a time of a few minutes to a few hours, one or more strong acids are introduced. The strong acid is advantageously hydrofluoric acid, alone or as a mixture with other strong acids, such as nitric acid, hydrochloric acid or trifluoromethanesulfonic acid. The wall then gradually hardens. The pH falls to the vicinity of 1 (indeed even 0.8) to 4, preferably of 1.5 to 2.5.

After one to a few hours, the temperature has risen, gradually or otherwise, up to the vicinity of 65° C. The temperature should be sufficiently high and the time sufficiently long for the alcohol produced by the reaction to be able to be largely removed by evaporation, given that this reaction is partially reversible. This temperature can vary from 40 to 100° C.

During this phase, the number of OH groups decreases in the body of the wall and at the surface of the microcapsules. The microcapsules may then become hydrophobic and may agglomerate, despite the presence of the protective colloid.

In order to overcome this, it is advantageous to introduce a hydrophilic silane which will be grafted to the surface of the microcapsules in order to render them permanently hydrophilic.

The silane suitable for the present invention is, for example, of the R₅—Si(R₁R₂R₃) or R₅Si—[(CH₃)R₁R₂] type
where R₅ is a nonhydrolyzable hydrophilic group, such as a poly(glycol ether), an epoxide group (capable of opening to give an OH, given the pH conditions) or a group carrying one or more acid, alcohol or amine functional groups. Among the silanes carrying one or more amine functional groups, an advantageous family is that comprising a cationized amine as it makes it possible to confer a cationic charge on the microcapsules which is very useful in cosmetic or textile applications, for example for the affinity for the skin or textile fibers which this charge confers;
and where the R₁, R₂ and R₃ groups are the hydrolyzable groups described above.

This silane compound is introduced after partial solidification of the wall, so that it remains at the surface and not in the body of the wall being formed, that is to say that it is introduced immediately before a tendency to agglomerate (which is reflected by a change in viscosity) appears.

Metal or organometallic catalysts well known to a person skilled in the art can be used to help in terminating the polymerization reaction, such as tin-comprising compounds, for example dibutyltin dilaurate, dibutyltin diacetate, tin octanoate, inorganic tin salts and platinum, zinc, zirconium, aluminum or titanium compounds, including titanates, for example.

Raising the pH:

This operation is not obligatory but, as the final pH of the microcapsules generally lies between 0.8 and 3.5 at the end of encapsulation, it is difficult to use them in this form. The pH is thus raised to approximately 6.5 for practical reasons and for reasons of compatibility with the media in which the capsules are used (the pH can range from 4 to 8.5 approximately). This operation is carried out with sodium hydroxide, potassium hydroxide or amines.

b) Water-In-Oil Encapsulation:

Preparation of the Aqueous Internal Phase:

The hydrophilic internal phase is prepared from hydrophilic active principles, such as proteins or protein hydrolyzates, amino acids (hydroxyproline, proline), polyols, such as glycerol, sorbitol, butylene glycol, propylene glycol or polyethylene glycol, allantoin, DHA, guanosine, sugars and sugar derivatives, water-soluble vitamins, such as ascorbic acid (vitamin C), hydroxy acids and their salts, and specific water-soluble active principles, such as moisturizing active principles, antiwrinkle agents, slimming agents, nutritional agents, softening agents, and the like.

Water necessary for the hydrolysis and polymerization reactions is necessarily added to these hydrophilic active principles, along with optionally a water-soluble solvent (for example glycol, alcohol, their ethers, their esters, glycerol, and the like).

Generally, all solvents which form a solution with water but which are not soluble in the lipophilic continuous phase may be suitable.

The active principle or principles are mixed or dissolved therein.

One or more weak or strong acids are dissolved therein, and optionally hydrofluoric acid or a water-soluble fluoride, so as to reduce the pH. It is possible to bring down the pH to, for example, between 1 and 4 from the stage of hydrolysis of the precursor.

It is also possible to introduce therein a silsesquioxane precursor compound as defined above. MTMS or MTES is preferably suitable.

The combined mixture is then stirred until the silsesquioxane precursors have sufficiently hydrolyzed to become soluble, before the emulsification operation.

Preparation of the Lipophilic Continuous Phase:

The continuous phase is an organic phase composed of esters, hydrocarbons, oils, silicone fluid, solvents or their mixtures and generally of any medium which is immiscible with water and liquid under the encapsulation conditions.

It is also possible to add a silsesquioxane precursor. Thus, this precursor can be present in one of the two internal or continuous phases or in both simultaneously.

Just as for the water-in-oil encapsulation, it is possible to add, to the lipophilic phase, an organosilicate, such as poly(ethyl silicate), which is insoluble in water even in the hydrolyzed state.

Manufacture of the Emulsion/Dispersion, Hydrolysis and Beginning of Polymerization:

As for the oil-in-water encapsulation, the addition of the internal phase takes place with stirring. The stirring speed is regulated in order to obtain the desired diameter.

The internal phase is generally present at a level of 40 to 45% of the mixture of the microcapsules.

An emulsifier as defined above can be added, preferably to the organic phase.

In an alternative form, a precursor compound of silsesquioxane type (MTES or MTMS) and optionally poly(ethyl silicate) can be introduced at this stage, if this has not already been done.

Under these conditions, the polymerization grows and the polymer chains lengthen.

Continuation and acceleration of the polymerization:

After a time of 30 min to a few hours, a lipophilic amine, such as tributylamine or dimethylbenzylamine, can be introduced with the aim of forming a complex with a strong acid, such as hydrofluoric acid, of the aqueous phase. If this acid is not present from the start in the aqueous phase, it is possible to react the amine with the hydrofluoric acid separately and to introduce the mixture obtained into the organic phase, after the phase of the start of hydrolysis/polymerization. It is also possible to do without the amine by introducing, with the hydrofluoric acid, into the aqueous phase, a fluoride, such as sodium fluoride or potassium fluoride.

The three-dimensional polymer is finally polymerized in its entirety in an acidic medium. The addition of the fluoride ions, by virtue of the hydrofluoric acid or of compounds comprising fluoride ions in their structure, makes it possible to promote the polycondensation of the silanol groups remaining free in the mixture.

The starting temperature is ambient temperature but it is possible to begin at higher temperatures. The final temperature lies between 40 and 80° C.

The wall is liquid at the start and gradually solidifies (in particular after introduction of the amine).

It is possible to introduce a lipophilic silane which will be grafted to the surface of the microcapsules in order to render them more lipophilic. This silane can be butyltrimethoxysilane or butyltriethoxysilane. This silane is introduced after partial solidification of the wall, so that it remains at the surface and not in the body of the wall being formed. In practice, it is introduced immediately before a tendency to agglomerate appears, which tendency is reflected by a change of viscosity.

It is also highly advantageous to introduce, into this polymerization phase, a silane carrying amine functional groups, at least one of which is cationized. This is because this results in microcapsules carrying a cationic charge. This type of surface modification greatly improves the possibilities of emulsification of the organic mixture of microcapsules in water, which is advantageous in numerous applications, including textiles. Here again, it is possible to add a metal catalyst as described above in order to accelerate the reactions.

Raising the pH:

This operation is not obligatory either but it is possible to raise the pH of the internal phase by introducing a base (mainly organic amine) into the organic phase, so as to obtain a pH of between 5.5 and 8.5.

Subsequently, the microcapsules comprising a water-in-oil or oil-in-water emulsion or dispersion can subsequently be dried in a spray tower or on a fluidized bed or by freeze drying or any other equivalent means.

In order to obtain leaktight microcapsules, it is necessary for the wall to be compact and nonporous. As described above, this can be obtained by polymerizing the wall very gradually, so that it remains liquid for as long as possible and solidifies only at the end of the operation by increasing the molecular weight and crosslinkings.

In order to give a better understanding of the subject matter of the invention, embodiments will be described as purely illustrative and nonlimiting examples of the scope of the invention.

### EXAMPLES

### Example 1

### Polymethylsilsesquioxane Microcapsules Comprising a Cosmetic Active Principle

70 g of tap water, 1.4 g of 40% citric acid and 16.0 g of a pyrrolidone/vinyl acetate copolymer (Collacral VAL from BASF) are introduced with stirring into a 500 cm³ beaker maintained at 40° C.

The stirring speed is increased and then a mixture of 86 g of Lipex 205 shea oil (sold by Unipex) and 0.72 g of tributylamine is introduced, in order to be emulsified, followed by 40 g of MTES (Dynasylan MTES from Degussa). After 40 min at 40° C., the following mixture is added: 12 g of 6% PEG-14M in water (molecular weight of 300 000 to 400 000) from Bisynthis, 3.0 g of 20% trifluoromethanesulfonic acid in water and 9.2 g of 20% hydrofluoric acid in water.

The temperature is maintained at 40° C. for 2 h and the stirring is regulated in order to obtain a microcapsule diameter of 20 μm.

4.0 g of glycidoxypropylmethyldiethoxysilane (Wetlink from Momentive) are then introduced in order to retain the hydrophilicity of the microcapsules. The temperature is then raised to 65° C. and maintained for 12 h, additions of water being carried out in order to maintain the level, which falls as a result of the evaporation (loss of alcohol and of water).

The emulsion is slowly cooled to 25° C. The pH is subsequently slowly raised to 6 with a 30% aqueous sodium hydroxide solution.

### Example 2

### Polymethylsilsesquioxane Microcapsules Comprising a Cosmetic Active Principle

35 g of tap water, 2.5 g of 40% citric acid, 1.5 g of 20% trifluoromethanesulfonic acid, 1.0 g of 20% hydrochloric acid, 6.0 g of a pyrrolidone/vinyl acetate copolymer (Collacral VAL from BASF), 15.0 g of MTES and 0.5 g of 3-aminopropylmethyldiethoxysilane (Dynasylan 1505 from Degussa) are introduced with stirring into a 300 cm³ beaker maintained at 40° C.

The stirring speed is increased and then the mixture of 43 g of olive oil squalene, 5 g of MTES and 0.36 g of tributylamine, brought to 50° C. and homogenized beforehand, is introduced, in order to be emulsified. The stirring is regulated in order to obtain a diameter of 15 μm.

After 15 min, the following mixture is added: 4 g of 6% solution of PEG-14M in water (molecular weight of 300 000 to 400 000) from Biosynthis and 4.6 g of 20% hydrofluoric acid in water.

The temperature is maintained at 40° C. for 1 h 30. 2.0 g of glycidoxypropylmethyldiethoxysilane (Wetlink 78 from Momentive) are introduced in order to retain the hydrophilicity of the microcapsules.

The temperature is then raised to 65° C. and maintained for 12 h, additions of water being carried out in order to maintain the level, which falls as a result of the evaporation (loss of alcohol and of water).

The emulsion is slowly cooled to 25° C. The pH is slowly raised to 6.0 with a 30% aqueous sodium hydroxide solution.

### Example 3

### Polymethylsilsesquioxane Copolymer Micro-Capsules Comprising a Fragrance

168 g of tap water, 1.4 g of 65% acetic acid and 77.0 g of MTMS (Dynasylan MTMS from Degussa) are introduced with stirring into a 800 cm³ reactor maintained at 25° C.

The mixture is stirred at 25° C. for 20 min.

The mixture of 14 g of tap water, 7 g of 20% trifluoromethanesulfonic acid in water and 21 g of 20% hydrofluoric acid in water is then added.

The following are then added with more vigorous stirring in order to manufacture the emulsion:

1) the mixture of 196 g of Rose Freesia 07 006 02 fragrance (Expressions Parfumées), 17.5 g of tripropylene glycol n-butyl ether (Dowanol TPnB from Dow), 56 g of poly(ethyl silicate) (Dynasil 40 from Degussa) and 2 g of tributylamine;

2) 17.5 g of a pyrrolidone/vinyl acetate copolymer (Collacral VAL from BASF).

The temperature is maintained at 25° C. for 1 h 30, then at 40° C. for 2 h and then at 75° C. for 30 min. During this time, the stirring is regulated in order to obtain a diameter of 6 μm.

12.0 g of Wetlink 78 (from Momentive) are introduced in order to retain the hydrophilicity of the microcapsules.

The temperature is maintained at 75° C. for 3 h 30, additions of water being carried out in order to maintain the level, which falls as a result of the evaporation (loss of alcohol and of water).

The emulsion is slowly cooled to 25° C. 16 h later, the pH is slowly raised to 6.5 with a 30% aqueous sodium hydroxide solution.

### Example 4

### Polymethylsilsesquioxane Copolymer Micro-Capsules Comprising a Phase Change Material (PCM)

440 g of tap water, 5.5 g of 65% acetic acid and 357.5 g of MTMS (Dynasylan MTMS from Degussa) are introduced with stirring into a 2.5 liter reactor maintained at 35° C.

The mixture is stirred at 35° C. for 20 min.

412 g of an 8% solution of carboxylated PVA in tap water (Poval KL318 from Kuraray) are then added. The mixture composed of 770 g of the active principle RT31 (paraffin wax melting at 31° C. from Rubitherm) mixed beforehand with 192 g of poly(ethyl silicate) (Dynasil from Degussa) and brought to 35° C. is then slowly introduced and the mixture is emulsified.

The mixture of 13.75 g of 20% trifluoromethanesulfonic acid in water, 35.75 g of 20% hydrofluoric acid in water and 55 g of tap water is then added.

The speed of the stirrer is regulated in order to obtain a diameter of 6 μm and the combined mixture is maintained at 35° C. for 3 h.

6.9 g of tributylamine are then added and the mixture is maintained at 35° C. for 1 h. It is then heated at 45° C. for 1 h 30 and subsequently at 75° C. for 3 h.

It is allowed to cool and, on the following day, the pH is raised to 6.0 with 30% aqueous sodium hydroxide solution.

### Example 5

### Polymethylsilsesquioxane Copolymer Micro-Capsules Comprising an Aqueous Active Principle

220 g of isononyl isononanoate, 879 g of cyclopentasiloxane, 161 g of poly(ethyl silicate) (Dynasil 40 from Degussa) and 4.4 g of cetyl dimethicone copolyol (Abil EM 90 from Goldschmidt) are introduced with stirring into a 3 liter jacketed vessel.

Once this continuous phase is homogeneous, the aqueous phase composed of the mixture of 988 g of a 30% aluminum sulfate solution, 29.3 g of 20% hydrofluoric acid in water and 11.7g of 50% AMP (2-amino-2-methyl-1-propanol) in water will be dispersed therein with vigorous stirring.

190 g of MTMS (Dynasylan MTMS from Degussa) are then introduced into the emulsion.

The mixture is maintained at 25° C. for 1 hour, then at 40° C. for 2 h and then at 60° C. for 2 h. The stirring is regulated in order to obtain a diameter of 20 μm.

7.3 g of triethanolamine are then introduced, followed by 3.5 g of dibutyltin diacetate.

The mixture is maintained at 60° C. for 4 h and is then allowed to cool.

### Example 6

### Polymethylsilsesquioxane Copolymer Micro-Capsules Comprising an Aqueous Active Principle

40 g of isononyl isononanoate, 40 g of 2-ethylhexyl cocoate, 10 g of cyclopentasiloxane, 9.6 g of poly(ethyl silicate) (Dynasil 40 from Degussa), 0.2 g of triethylamine and 1 g of cetyl dimethicone copolyol (Abil EM 90 from Goldschmidt) are introduced with stirring into a 350 ml beaked immersed in a water bath at 20° C.

Once this continuous phase is homogeneous, the prehomogenized aqueous phase consisting of 74 g of “Fleur de bach” (aqueous extract), 3.0 g of 20% hydrofluoric acid in water and 0.2 g of triethylamine will be dispersed therein with vigorous stirring.

20 g of MTMS (Dynasylan MTMS from Degussa) are then introduced into the emulsion.

The temperature is maintained at 20° C. for 2 hours and then at 40° C. for 2 h. The stirring is regulated in order to obtain a diameter of 8 μm.

2.0 g of a cationic amino silane (Dynasylan 1172 from Degussa) are then introduced. The mixture is then maintained at 40° C. for 4 h. The organic mixture of microcapsules obtained can be easily emulsified in water due to the cationic charges attached to the microcapsules.

### Example 7

### Polymethylsilsesquioxane Copolymer Micro-Capsules Comprising an Aqueous Active Principle

12.30 g of isononyl isononanoate, 49.1 g of cyclopenta-siloxane, 9.9 g of poly(ethyl silicate) (Dynasil 40 from Degussa) and 0.3 g of cetyl dimethicone copolyol (Abil EM 90 from Goldschmidt) are introduced with stirring into a 250 ml jacketed beaker.

60.7 ml of a 50% solution of glutaraldehyde in water are mixed with 1.8 g of a 20% hydrofluoric acid solution in a 100 ml beaker. 1.4 g of MTES (Dynasylan MTES from Degussa) are dispersed in this mixture at ambient temperature.

After 15 min, the aqueous phase becomes transparent. It is then emulsified with vigorous stirring in the preceding organic phase and then 12.5 g of MTMS (Dynasylan MTMS from Degussa) are introduced into the emulsion.

The mixture is maintained at ambient temperature for 1 hour, during which the stirring is regulated so as to obtain a diameter of 8 μm, and then 0.5 g of tributylamine is introduced.

The mixture is then heated at 40° C. for 2 h and then at 60° C. for 1 h.

0.1 g of dibutyltin diacetate is subsequently introduced.

The mixture is maintained at 60° C. for 2 h, in order to bring the reaction to completion, and is then allowed to cool.

### Examples 1 to 7 make it possible to obtain reservoir microcapsules, the wall of which is formed of silsesquioxane, which are leaktight and resistant.

Although the invention has been described in connection with a specific embodiment, it is clearly evident that it is in no way limited thereto and that it comprises all the technical equivalents of the means described and their combinations, if the latter come within the scope of the invention.

What is claimed is:

1. A process for the manufacture of reservoir microcapsules, said microcapsules comprising a core of at least one active principle surrounded by a polymer shell, said polymer shell comprising 50% to 100% by weight of a silsesquioxane compound with respect to the total weight of said shell, the process comprising:
dispersing at least one hydrophilic active principle in an organic continuous phase, so as to form a water-in-oil emulsion or dispersion;
introducing into the organic phase (i) a precursor of the silsesquioxane polymer compound, and (ii) a poly(ethyl)silicate that is insoluble in water in the hydrolyzed state;
hydrolyzing and polymerizing said precursor in situ in or on contact with the aqueous phase of the water-in-oil emulsion or dispersion, so as to form a silsesquioxane homopolymer or copolymer, wherein said polymerizing is carried out at a pH of less than 6.

2. The process according to claim 1, wherein the (i) precursor is introduced into the organic phase and hydrolyzed, before the (ii) organo-silicate is introduced into the organic phase, and then the hydrolyzed precursor is polymerized.

3. The process according to claim 1, wherein the silsesquioxane polymer compound is of formula R—SiO_{3/2}, where R is a substituted or unsubstituted C1-C20 alkyl radical, an alkoxy alkyl radical, a halogenated radical, or an unsaturated radical.

4. The process according to claim 1, wherein said hydrolyzing is carried out at a pH of from 3 to 5.

5. The process according to claim 6, wherein said polymerizing begins at an initial pH between 3 to 5, and proceeds at a lower pH up to the end of the polymerization reaction of from 1 to 4.

6. The process according to claim 5, wherein the lower pH is from 1.5 to 2.5.

7. The process according to claim 1, wherein said hydrolyzing and said polymerizing are carried out at a pH of from 1 to 4.

8. The process according to claim 1, wherein said polymerizing is carried out in the presence of fluoride ions or one or more compound comprising fluoride ions.

9. The process according to claim 8, wherein said one or more compound has an amine functional group.

10. The process according to claim 1, wherein the pH at the end of the polymerizing reaction is raised to between 5.5 and 8.5.

11. The process according to claim 1, further comprising introducing one or more silane carrying a lipophilic group into the organic phase.

12. The process according to claim 14, wherein the one or more silane carries a cationic charge.

13. The process according to claim 1, wherein the temperature is from 10° C. to 50° C. during the dispersing or hydrolyzing, and is from 40° C. to 90° C. during the polymerizing.

14. The process according to claim 1, wherein the precursor of the silsesquioxane polymer compound is of formula R—Si(R₁R₂R₃), wherein R is a substituted or unsubstituted C1-C20 alkyl radical, an alkoxy alkyl radical, a halogenated radical, or an unsaturated radical; and
R₁, R₂ and R₃ each independently is an acetoxy, amino, acid, amide, oximino, chlorine or OR₄ group, where R₄ is a substituted or unsubstituted C1-C3 alkyl radical, an alkoxy alkyl radical, or an unsaturated radical.

15. The process according to claim 1, wherein the precursor of the silsesquioxane polymer compound is methyltrimethoxysilane (MTMS), methyltriethoxysilane (MTES), methyltrichlorosilane, or mixtures thereof.

16. The process according to claim 1, wherein the polymer shell comprises 70% or more by weight of the silsesquioxane compound with respect to the total weight of the shell.

17. The process according to claim 1, wherein the at least one hydrophilic active principle is selected from the group consisting of proteins or protein hydrolysates, amino acids, polyols, glycerol, sorbitol, butylene glycol, propylene glycol, polyethylene glycol, allantoin, DHA, guanosine, sugars and sugar derivatives, water-soluble vitamins, ascorbic acid, and hydroxy acids and their salts.

18. The process according to claim 1, wherein the at least one hydrophilic active principle is selected from the group consisting of moisturizing active principles, antiwrinkle agents, slimming agents, nutritional agents, and softening agents.

19. The process according to claim 3, wherein R is a substituted or unsubstituted C1-C20 alkyl radical selected from the group consisting of: methyl, ethyl, n-propyl, isopropyl, 1-n-butyl, 2-n-butyl, isobutyl, tert-butyl, n-pentyl, isopentyl, neopentyl, tert-pentyl, hexyl, n-hexyl, heptyl, n-heptyl, octyl, n-octyl, isooctyl, 2,2,4-trimethylpentyl, nonyl, decyl, dodecyl, octadecyl, cycloalkyl, cyclopentyl, cyclohexyl, cycloheptyl, methylcyclohexyl, aryl, phenyl, naphthyl, anthrylm, phenanthryl, alkaryl, o-tolyl, m-tolyl, p-tolyl, xylyl, ethylphenyl, aralkyl, benzyl, α-phenylethyl and β-phenylethyl radicals.

20. The process according to claim 3, wherein R is
an alkoxy alkyl radical, methoxyethyl or ethoxyethyl;
a halogenated radical selected from the group consisting of chloropropyl, 3,3,3-trifluoro-n-propyl, 2,2,2,2′,2′,2′-hexa-fluoro-isopropyl, heptafluoroisopropyl, o-chloro-phenyl, m-chloro-phenyl, and p-chloro-phenyl; or
an unsaturated radical selected from the group consisting of vinyl, 5-hexenyl, 2,4-divinylcyclohexylethyl, allyl, 3-butenyl, 4-pentenyl, ethynyl and propargyl.
